(19) United States
(12) Patent Application Publication
(10) Pub. No.: US 2009/0197947 A1
(43) Pub. Date: Aug. 6, 2009

(54) MEDICAMENTS AND METHODS FOR LOWERING PLASMA LIPID LEVELS AND SCREENING DRUGS

(75) Inventors: Mohammed Mahmood Hussain, Woodbury, NY (US); Jahangir Iqbal, Jersey City, NJ (US)

Correspondence Address: HOFFMANN & BARON, LLP, 6900 JERICHO TURNPIKE, SYOSSET, NY, 11791, US

(73) Assignee: The Research Foundation of State University of New York, Albany, NY (US)

(21) Appl. No.: 12/362,496

(22) Filed: Jan. 30, 2009

Related U.S. Application Data
(60) Provisional application No. 61/025,562, filed on Feb. 1, 2008.

(51) Int. Cl. A61K 31/366; C12Q 1/60

(52) U.S. Cl. 514/460; 435/11

(57) ABSTRACT

The invention relates to compositions, medicaments, methods for treating individuals with high plasma lipid levels, and methods for screening drug candidates useful in, for example, the treatment of hypercholesterolemia. Specifically, the invention relates to the discovery that MTP inhibition leads to increased accumulation in cellular free-cholesterol and is useful in the development of compositions, medicaments, methods of treatment and drug screening methods to treat high plasma lipid levels.

### CROSS-REFERENCE TO RELATED APPLICATIONS

The present invention claims priority to U.S. Provisional Application No. 61/025,562, filed Feb. 1, 2008, the entire contents of which are incorporated herein by reference.

### FUNDING STATEMENT

This invention was made with government support under contract identifier DK46900 and HL64272 awarded by the National Institute of Health. The government has certain rights in the invention.

### FIELD OF THE INVENTION

The invention relates to compositions, medicaments, methods for treating individuals with high plasma lipid levels, and methods for screening drug candidates useful in, for example, the treatment of hypercholesterolemia. Specifically, the invention relates to the discovery that MTP inhibition leads to increased accumulation in cellular free-cholesterol and is useful in the development of compositions, medicaments, methods of treatment and drug screening methods to treat high plasma lipid levels.

### RELATED ART

Though there are many different types of cholesterol lowering medicines available to help subjects maintain a healthy level of LDL cholesterol, some of the more popular classes of compounds (e.g. statins) may have serious adverse side effects, or may be ineffective at treating subjects with certain conditions and/or diagnoses. Microsomal Triglyceride Transfer Protein (MTP) inhibitors were developed by many pharmacological institutions in efforts to combat cholesterol related ailments and diagnoses with a new type of treatment. In clinical trials, each of the various MTP inhibitor agents was toxic to the liver and resulted in damage and destruction of tissue usually associated with increased plasma levels of aminotransferases. Therefore, it is well known and accepted that MTP inhibition leads to liver damage. It has been generally accepted that the tissue damage might be secondary to the accumulation of neutral lipids, such as triglycerides and cholesterol esters.

MTP and its various characteristics and uses are disclosed in Applicants' publications, including Hussain et al., “Microsomal Triglyceride Transfer Protein: A Multifunctional Protein,” Frontiers in Bioscience 8, s500-506 (2003); Hussain et al., “Microsomal triglyceride transfer protein and its role in apoB-lipoprotein assembly,” J. Lipid Research, Vol. 44, pp. 22-32 (2003); Hussain et al., “Microsomal triglyceride transfer protein in plasma and cellular lipid metabolism,” Curr. Opin. Lipidol, Vol. 19, pp. 277-284 (2008); and Hussain et al., “New approaches to target microsomal triglyceride transfer protein,” Curr. Opin. Lipidol, Vol. 19, pp. 572-578 (2008), the entire contents of each publication are incorporated by reference herein.

The present invention seeks to provide an effective and safe method to treat individuals having an undesirable cholesterol level while avoiding the serious drawbacks and complications of prior methods and compositions.

### SUMMARY OF THE INVENTION

In one aspect of the present invention, there is provided a composition for treating high plasma lipid levels, the composition including at least one MTP inhibitor, at least one of a cholesterol efflux enhancer and a cholesterol synthesis inhibitor, and at least one bile acid synthesis enhancer.

Another aspect of the present invention provides a medicament for a subject having high plasma lipid levels, having a therapeutically effective amount of MTP inhibitor, a therapeutically effective amount of at least one of a cholesterol efflux enhancer and a cholesterol synthesis inhibitor, a therapeutically effective amount of bile acid enhancer, and a biologically compatible carrier.

Still another aspect of the present invention provides a method of treating a subject with hypercholesterolemia, the method including the steps of administering to the subject an effective amount of a composition for treating high plasma lipid levels having at least one MTP inhibitor; and at least one of (1) a cholesterol efflux enhancer and (2) a cholesterol synthesis inhibitor. Optionally, the composition may include a bile acid synthesis enhancer.

In still other embodiments of the present invention, there is provided a method of treating an individual, including the steps of inhibiting MTP in cells of the individual, decreasing cholesterol synthesis in cells of the individual, and/or enhancing conversion of cholesterol to bile acids in cells of the individual and/or enhancing free cholesterol efflux in cells of the individual.

Still yet another aspect of the present invention provides: an in vitro method of screening for effective and safe MTP inhibitors for administration to an individual, the method including the steps of measuring a first free-cholesterol level of at least one liver or intestine derived cell of an individual, administering a candidate MTP inhibitor to cell, measuring a second free-cholesterol level of the cell, and determining whether the second free-cholesterol level is greater than the first free-cholesterol level, where increase of free-cholesterol level is indicative of an effective MTP inhibitor for the individual.

A further aspect of the present invention provides: a method of screening potential cholesterol efflux enhancers for administration to an individual including the steps of: taking a cell sample from the individual; inhibiting MTP from the cell sample to accumulate a first free-cholesterol count; measuring the first free-cholesterol count in the cell sample; administering a candidate free-cholesterol efflux enhancer to the cell sample; measuring a second free-cholesterol count in the cell sample; and determining whether the second free-cholesterol count is lower than the first free-cholesterol count; where decrease of free-cholesterol count is indicative of an effective free-cholesterol efflux enhancer for the individual.

Even further, the present invention provides: a method for screening candidate cholesterol efflux enhancing drugs, comprising: inhibiting MTP from a cell sample to accumulate a free-cholesterol count; administering a candidate free-cholesterol efflux enhancer to the cell sample; and assaying the cell sample to determine a necrotic count of the cell sample and correlate necrosis to an effective free-cholesterol efflux enhancer. It should be noted that using liver derived cells increases in liver enzymes in the media could also be used as a measure to assess liver injury. These experiments can also be performed in vivo. Under such conditions, increases in liver enzymes can be used to monitor hepatic injury due to MTP inhibition as well as their decreases as a sign of successful treatment.

These and other features of the invention will be better understood through a study of the following description and appendixes.

### BRIEF DESCRIPTION OF THE FIGURES

FIG. 1 depicts the normal pathway of lipid production and circulation of an individual.

FIG. 2a depicts the expected results of the pathway after MTP inhibition.

FIG. 2b depicts the results of the pathway after MTP inhibition.

FIG. 3a depicts the effect of a combination of MTP inhibition and cholesterol biosynthesis inhibition.

FIG. 3b depicts the effect of a combination of MTP inhibition and cholesterol efflux enhancement.

FIG. 3c depicts the effect of a combination of MTP inhibition and bile acid synthesis activation.

FIG. 3d depicts the effect of a combination of MTP inhibition, cholesterol biosynthesis inhibition, and fatty acid oxidation activation.

FIG. 4 sets forth the results of an experiment to determine the effect of efflux enhancer and cholesterol biosynthesis inhibitor on plasma lipids.

FIG. 5 sets forth the results of an experiment to determine effect of efflux enhancer and cholesterol biosynthesis inhibitor on hepatic mRNA, MTP activity and lipid levels.

FIG. 6 sets forth the results of an experiment to determine the effect of mttp gene deletion on plasma lipids and lipoproteins.

FIG. 7 sets forth the results of an experiment to determine intestinal and hepatic cholesterol levels after MTP inhibition.

FIG. 8 sets forth the results of an experiment to determine hepatic and intestinal expression of MTP and ACAT after MTP inhibition.

FIG. 9 sets forth the results of an experiment to determine whether chemical inhibition of MTP decreases cholesteryl esters.

FIG. 10 sets forth the results of an experiment to determine whether overexpression of MTP is sufficient to increase cholesteryl ester levels.

FIG. 11 sets forth the results of an experiment to determine whether inhibition of cholesteryl ester synthesis by cholesteryl esters is alleviated by MTP.

### DETAILED DESCRIPTION OF THE INVENTION

The present invention relates to methods and compositions for preventing the excessive amount of free-cholesterol accumulation in the cells of an animal, in particular humans, by reducing the amount of MTP in a cell. In particular, the amount of MTP may be reduced, for example, by administering a pharmaceutically effective amount of an MTP inhibitor. As used herein, the term “excessive amount” refers to a level of free-cholesterol which is toxic or otherwise deleterious to the health of the cell. Such excessive accumulation can lead to cell damage or necrosis. Such cells may include any cells in the individual, and in particular include cells in the liver or intestine of the individual.

Embodiments of the present invention may include a combination therapy and a therapeutic composition. Such compositions may collectively perform at least one of the following: inhibit MTP (to decrease lipoprotein biosynthesis), decrease cholesterol synthesis, enhance conversion of cholesterol to bile acids and enhance free cholesterol efflux to avoid tissue damage to the liver. As such, the present invention provides an effective and safe treatment option for many of the conditions unresponsive to other treatments or patients with adverse side effects to the other treatments. In addition, various embodiments of the present invention employ MTP inhibitor determination and plasma lipid and liver enzyme measurements as the basis for testing individual or combination candidate drug efficacy in treating various related conditions and diagnoses.

It has been shown that MTP inhibition leads to decreased accumulation of cholesterol esters and increased amounts of free-cholesterol. While it was generally assumed previously, that MTP inhibitor induced tissue damage was due to the accumulation of neutral lipids (e.g. triglycerides and cholesterol esters); the various embodiments of the present invention are based upon the determination that liver damage due to MTP inhibition may result from the accumulation of free cholesterol in the liver. MTP inhibition may be achieved through genetic ablation or chemical inhibition, e.g., through administration of MTP antagonists or inhibitors.

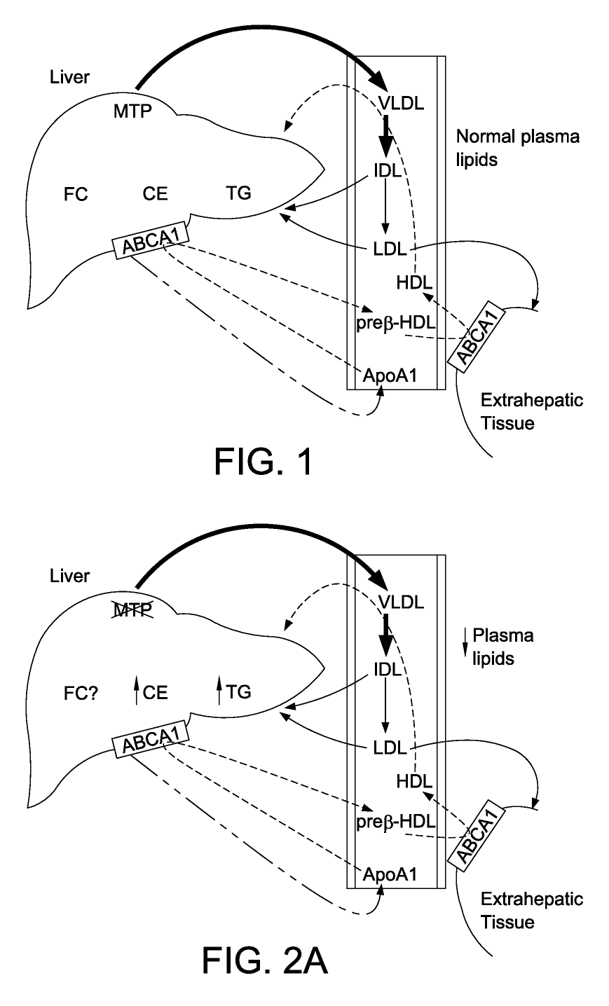

With reference to FIG. 1, a normal pathway of the liver is depicted. In this normal pathway, MTP assembles and secretes neutral lipid (triglyceride and cholesteryl ester) rich very-low density lipoprotein (VLDL) from the liver into the circulation where it is acted upon by lipases to form intermediate density lipoprotein (IDL). Most of the IDL is taken up by the liver, but some of it is converted into low-density lipoprotein (LDL) by the action of lipoprotein lipases. LDL from the circulation is taken up by the liver and other extrahepatic tissues by receptor mediated endocytosis. Free cholesterol, on the other hand, is effluxed by membrane bound ATP binding cassette transporter 1 (“ABCA1”) onto apoA1 to form preB-HDL, which in turn gets converted into HDL by accepting free cholesterol from the tissues. HDL delivers cholesterol back into the liver. This process is referred to as “reverse cholesterol transport.”

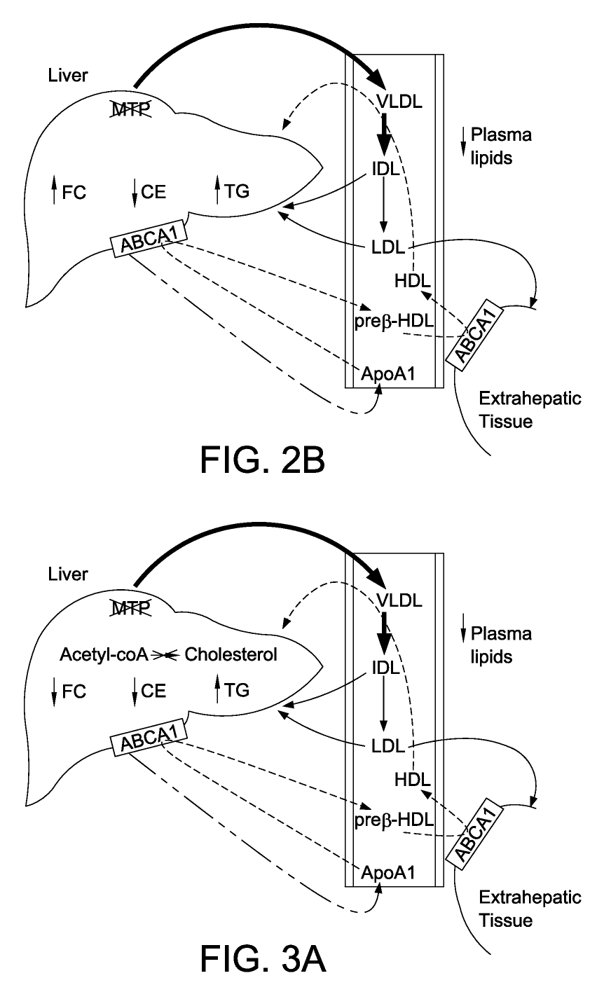

FIG. 2a depicts the pathway to be expected after MTP inhibition. Inhibition of MTP prevents assembly of neutral lipids onto apoB-containing lipoproteins, and therefore leads to decreased VLDL secretion into the circulation. Furthermore, inhibition of MTP should lead to accumulation of neutral lipids TG and CE into the liver. However, as depicted in FIG. 2b and discussed in more detail below, the inventors have discovered that inhibition of MTP increases hepatic TG levels but surprisingly lowers CE levels in the liver. Decrease in CE synthesis leads to accumulation of FC.

New understanding that MTP plays a valid role in the biosynthesis of cholesterol esters helps to explain the toxicity previously associated with MTP inhibitors. The present applicants have discovered that the previously accepted notion—that MTP inhibitors are toxic because they lead to triglyceride accumulation—may not be totally accurate. Rather, the present inventors have discovered that MTP inhibition leads to the accumulation of free cholesterol as well. This high count of free cholesterol in patients is toxic to cells, and causes liver damage. Understanding the role that MTP plays in the biosynthesis of cholesterol esters aids in treating cholesterol related disorders, as well as screening candidate drugs as effective treatments.

The compositions and/or medicaments disclosed herein may be employed in therapies for treating and/or preventing cholesterol-related disorders and diagnoses including, inter alia, obesity, high plasma lipid levels, athosclerosis, hyperlipidemia, hypercholesterolemia, familial combined hyperlipidemia, familial hypercholesterolemia, and the like. Therapies treating these conditions may employ, for example, MTP inhibitors and at least one of (1) cholesterol efflux enhancers and/or (2) cholesterol synthesis inhibitors. Optionally, the compositions may include one or more bile acid synthesis enhancers. With any combination, it is possible to prevent cholesterol creation or increase the outflow of free cholesterol from the cells, preventing cell toxicity and necrosis. Various compounds and agents, which act as MTP inhibitors, cholesterol efflux enhancers, and cholesterol synthesis inhibitors are known and used in the art, however the particular combination of such components has previously been unknown and is now found to provide beneficial results.

In some embodiments, cellular samples from subject individuals may be genetically or chemically manipulated to inhibit MTP activity in order to screen and test new drug candidates, especially cholesterol efflux enhancers, cholesterol synthesis inhibitors, and/or bile acid synthesis enhancers individually or in combination with one or more known compounds and agents.

An aspect of the present invention provides a composition for treating high plasma lipid levels. In one embodiment, the composition includes a combination of various components working together. One combination provides a composition includes various combinations of the following: at least one MTP inhibitor, at least one cholesterol synthesis inhibitor, at least one bile acid synthesis enhancer, and at least one cholesterol efflux enhancer. The composition may include one or more fillers or biologically-acceptable carriers as is known in the art. The composition may include one or more types of one component, as may be desired. For example, two different MTP inhibitors may be used in combination. Similarly, an MTP inhibitor may be employed with a cholesterol efflux enhancer, a cholesterol synthesis inhibitor, a bile acid synthesis enhancer or all of them to provide the desired result in a subject or subject cells, dependent, in part, on the medical condition or diagnosis being treated.

Also, another aspect of the present invention provides a medicament for in vivo treatment of a subject having high plasma lipid levels. The medicament may comprise one or more of the various forms of the composition, in combination with a biologically compatible carrier. The carrier may be in one or more forms in order to expedite the administration of the composition to the in vivo area. That is, the carrier may be a solid, liquid, or gas, in one or more forms which may be contacted to subject cells. The medicament may be administered in any desired means so as to provide the medicament to the target cell of the individual, including via oral, intravenous, intranasal, intraperitoneal, intramuscular, intradermal or subcutaneous administration, by suppository or by infusion or implantation.

Another aspect of the present invention provides a method of treating a subject with hypercholesterolemia. Such a method may comprise, for example: administering to the subject a therapeutically effective amount of the composition for treating high plasma lipid levels having: at least one MTP inhibitor; and at least one of (1) a cholesterol efflux enhancer, (2) bile acid synthesis enhancer, and (3) a cholesterol synthesis inhibitor. As will be appreciated by one of skill in the art, one or more of the steps may be reiterated as may be needed to effectively prevent, treat, or ameliorate the condition.

An MTP inhibitor, as referred to herein, refers to a compound which inhibits the lipid transfer activity of MTP. As used herein, MTP refers to a heterodimeric protein that transfers neutral lipids between membranes in vitro. MTP has three main functions, including lipid transfer activity, apoB binding, and membrane association. An MTP inhibitor includes, for example, BMS 200150, BMS 197636, BMS 211221, CP-346086, and the like. The protein, MTP, transfers lipids onto ApoB, which results in the formation of lipoproteins. MTP is thus required for the biosynthesis of very low-density lipoprotein by the liver and chylomicrons by the intestine. Lipoproteins are present in the blood to transport lipids (i.e. triglycerides, cholesterol, phospholipids etc.). Ultimately, these apoB lipoproteins are converted to LDL (low-density lipoprotein) or chylomicron remnants. LDL is considered “bad cholesterol”, and has been linked to disease. Once LDL is formed, it accumulates in the plasma. Higher plasma LDL cholesterol levels are considered harmful.

Any desired and effective cholesterol synthesis inhibitors may be used in the present invention. Suitable cholesterol synthesis inhibitors include, for example, the statin class of compounds, including both fermentation derived and synthetic statins. Statins are commonly prescribed to patients with various cholesterol related diagnoses. Statins effectively shut down the synthesis of cholesterol.

Cholesterol efflux enhancers are compounds which can increase cholesterol output or outflow from the cell. Though cholesterol efflux enhancers are somewhat known, their effectiveness and use have not to this point been fully explored. Thus, a method for screening drug candidates and identifying successful candidates is needed.

Bile acid synthesis enhancers may be included in the novel compositions described herein. Any bile acid synthesis enhancer may be used, including but not limited to GW4064, MFA-1, and the like.

Compositions for treatment may include any desired amounts of MTP inhibitors, cholesterol synthesis inhibitors, cholesterol efflux enhancers, bile acid synthesis enhancers, and pharmaceutically acceptable carriers. In particular, if any particular component is used in the composition, that component is preferably present in a therapeutically effective amount. Depending on the particular amount of one component, there may be required more or less of the other component(s) (including zero). Finally, fillers, additives, and other materials, including pharmaceutically acceptable carriers may be present in any desired amount so as to complete the composition.

As explained above, MTP is a chaperone required for the synthesis of apoB-lipoproteins. So, MTP inhibitors in turn inhibit the synthesis of the ApoB containing lipoprotein (of which, LDL is a by-product). If MTP is inhibited, the synthesis of LDL is reduced. Statins increase the removal of LDL from plasma. In this respect, a combination of MTP inhibitors and cholesterol synthesis inhibitors are useful in preventing apoB lipoproteins from entering the system. At the same time, their removal from the system is enhanced. Such a combination therapy may be useful in decreasing plasma lipid levels and decreasing cellular free cholesterol levels. In this respect, the amounts of LDL or VLDL coming into the plasma are reduced. When a bile acid synthesis enhancer and/or a cholesterol efflux enhancer is included, the effectiveness of the composition may be increased. One beneficial result of administration of the present invention is to lower high plasma lipid levels by using agents with additive or combinatorial effects involving prevention of lipoprotein synthesis and enhancement of catabolism of LDL already in the system.

The catabolism/metabolism of cholesterol (lipoproteins) under one embodiment of the present invention may be explained by equating to changes in the water levels in a sink, the sink having an input tap and an output drain. Under steady state, the water coming from the tap pours water into the sink at a constant rate, while the drain removes water from the sink at a constant rate. An equilibrium between these two processes maintains the steady state levels of water in the sink. Cholesterol synthesis inhibitors (e.g. Statins) effectively decrease the inflow into the sink such that the plasma levels go down. This results in a decrease in plasma levels, which is beneficial. At the same time, MTP inhibitors may be used to slow the flow of tap water into the sink (by preventing synthesis of LDL), so again the plasma lipid levels decrease. In providing a combination therapy of cholesterol synthesis inhibitors and MTP inhibitors, such as set forth in the present invention, there is provided an additive effect on the lowering of plasma lipid levels. This may also lead to lower dosages or fewer iterations of the composition in order to acquire the desired result.

The composition may be administered to a subject for either prevention or treatment of a condition. In effect, administration of the therapy will lower the plasma lipid level of a patient with hyperlipidemia. Another important application of the present invention is as an essential treatment for patients with familial hypercholesterolemia. For patients with familial hypercholesterolemia, traditionally accepted cholesterol synthesis inhibitors (i.e. statins) have not been found to work effectively. Therefore, the present invention provides a treatment for, in particular, familial hypercholesterolemia patients who otherwise would ultimately need a liver transplant in order to promote successful lowering of plasma lipid levels.

Typically, in patients suffering from familial hypercholesterolemia, a composition comprising an MTP inhibitor plus a statin may be a valid treatment. However, with familial hypercholesterolemia, a composition comprising an MTP inhibitor and a cholesterol efflux enhancing compound would provide an even more effective treatment.

To determine if the drug is performing effectively in a subject or on subject cells, it may be necessary to measure the plasma and cellular cholesterol levels in the cells of the subject. Any known or desired means to measure the level of plasma and/or cellular cholesterol levels in the cells of a subject may be used. In some embodiments, for example, one may measure the plasma cholesterol levels and liver enzymes (surrogates for liver damage). In animal subjects in particular, it is possible to directly measure the changes in liver free cholesterol and other lipid levels, in order to determine the toxicity of liver tissue as a result of the accumulation of lipids in the liver. In addition, liver enzymes, including aminotransferases, may be measured as indicators of liver tissue injury.

By preventing free cholesterol accumulation, the present invention prevents liver injury and tissue damage. Therefore, if free cholesterol accumulation can be avoided, MTP inhibitors will not be cytotoxic, and will therefore have improved efficacy as a treatment. By reducing and/or eliminating free cholesterol accumulation, the present therapeutic composition may be used to treat hyperlipidemic subjects having high plasma lipid levels. The two major problems in the population that the composition and therapy of the present invention may treat include genetic disorders like familial hypercholesterolemia as well as environmental disorders such as obesity.

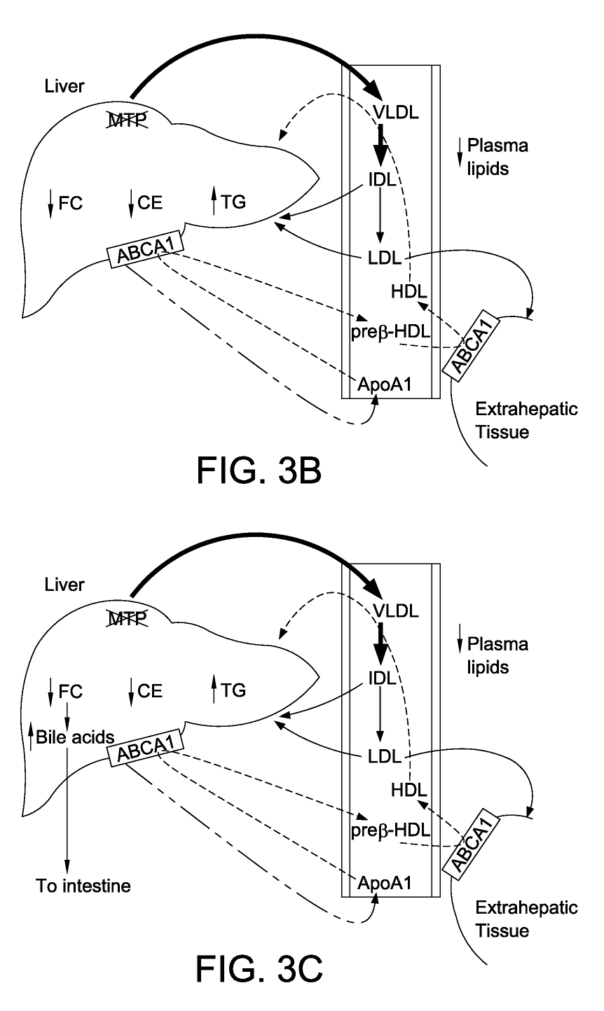

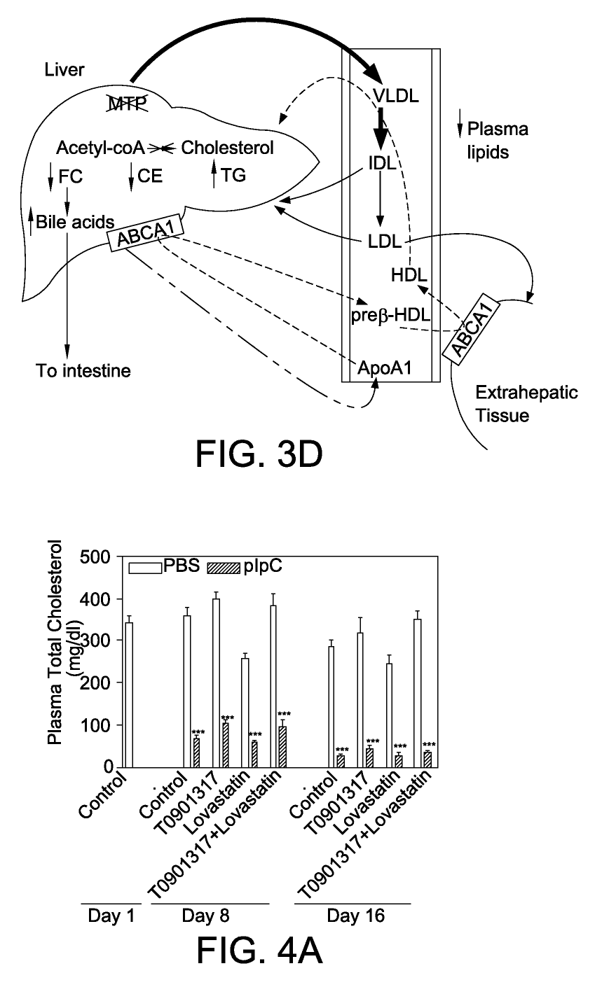

As depicted in FIG. 3a, a combination of MTP inhibitor and a cholesterol biosynthesis inhibitor is expected to decrease FC levels in the liver. As seen in FIG. 3b, the combination of an MTP inhibitor and a cholesterol efflux enhancer may decrease levels of FC in the liver by increasing its efflux into the circulation of the individual. Further, as depicted in FIG. 3c, the combination of an MTP inhibitor and a bile acid synthesis activator may also decrease levels of FC in the liver. This is accomplished by increasing the conversion into bile acids, which are subsequently excreted into the small intestine. Finally, as depicted in FIG. 3d, the combination of MTP inhibition, cholesterol synthesis inhibition, and fatty acid oxidation activation may decrease both FC and TG levels in the liver. Any combination of MTP inhibitor with any of the above may provide beneficial results to an individual.

Understanding the mechanism of the MTP chaperoning effect allows for the present mechanism to be employed as an assay for testing candidate drugs, i.e. cholesterol efflux enhancers, cholesterol synthesis inhibitors, and/or MTP antagonists/inhibitors. Such assaying may be performed to determine the most suitable cholesterol efflux enhancers, cholesterol synthesis inhibitors, and/or MTP antagonists/inhibitors for the particular patient being treated. In particular, the assaying method described herein may be performed in vitro, allowing for safer and less costly screening methods.

The present invention therefore provides an in vitro method of determining MTP inhibitors. The method of determining MTP inhibitors may include a series of steps to arrive at the most suitable MTP inhibitor. By the present method, one first cultivates a sample cell from the subject individual. As explained above, the subject cells may include, for example, cells extracted from the liver or the intestine of the individual. Once the cell has been cultivated, one may measure a first free-cholesterol level of at least one cell. That first free-cholesterol level is recorded. Next, a therapeutically effective amount of a potential MTP inhibitor is administered to the sample cell, and allowed to take effect on the cell. Once the cell has been exposed to the MTP inhibitor for a suitable time, a second free-cholesterol level measurement of the sample cell may be taken and recorded. The user then may compare the first and second free-cholesterol level measurements to determine whether the second free-cholesterol level is greater than the first free-cholesterol level. An increase in the level of free-cholesterol level after administration of the MTP inhibitor may be indicative of an effective MTP inhibitor.

Similarly, another aspect of the present invention provides a method of determining cholesterol efflux enhancers. The method of determining cholesterol efflux enhancers may include a series of steps to arrive at the most suitable cholesterol efflux enhancer. By the present method, one first cultivates a sample cell from the subject individual. As explained above, the subject cells may include, for example, cells extracted from the liver or the intestine of the individual. Once the cell has been cultivated, the user may inhibit MTP from a cell sample to accumulate a free-cholesterol count. That first free-cholesterol count may be recorded. Next, a therapeutically effective amount of a potential free-cholesterol efflux enhancer may be administered to the cell sample. Once the cell sample has been exposed to the free-cholesterol efflux enhancer for a suitable time, a second free-cholesterol count measurement of the sample cell may be taken and recorded. The user then may compare the first and second free-cholesterol count measurements to determine whether the second free-cholesterol count is lower than the first cholesterol count. A decrease in the level of free-cholesterol count after administration of the free-cholesterol efflux enhancer may be indicative of an effective free-cholesterol efflux enhancer.

In experiments focused on the inhibition of MTP activity and the assembly/secretion of apoB-lipoproteins, cellular lipid levels were measured. As will be discussed in the Examples below, MTP inhibition led to decreased accumulation of cholesterol esters and increased amounts of free cholesterol. In addition, it has been revealed that MTP enhances cholesterol esterification by removing cholesterol esters from the site of synthesis. These studies also showed that cholesterol esters inhibit cholesterol ester synthesis. MTP relieves this product inhibition by removing cholesterol esters from their site of synthesis and depositing them into apoB-lipoproteins. The experimentation led to knowledge that may be useful in avoiding toxicity associated with the use of MTP inhibitors to lower plasma lipid levels.

According to the studies conducted by the inventors and set forth below, reductions in the MTP activity achieved either via genetic ablation or chemical inhibition decrease cholesteryl esters and increase free cholesterol levels in intestinal and liver cells. These changes are not related to alterations in the cholesterol esterifying enzymes. Microsomes isolated from MTP deficient animals synthesized reduced amounts of cholesteryl esters and providing purified MTP ameliorated this deficiency. Furthermore, expression of MTP and apoB along with ACAT2 in cells that do not express these proteins were found to enhance cholesteryl ester synthesis. Further studies revealed that the enrichment of membranes with cholesteryl esters inhibits cholesteryl ester synthesis. In addition, transfer of cholesteryl esters to apoB-lipoproteins by MTP augments cholesteryl ester synthesis. Therefore, it can be determined that cholesteryl ester biosynthesis is inhibited by product accumulation, and MTP avoids this inhibition by transferring them to nascent apoB-lipoproteins.

A number of embodiments of the invention have been described herein. Nevertheless, it will be understood that various modifications may be made without departing from the spirit and scope of the invention. Also, the steps described in the methods above may be modified in various ways or performed in a different order than described above, where appropriate. Accordingly, alternative embodiments are within the scope of the disclosure. Various methods for inhibition of MTP may be found in Applicants' publication, Iqbal et al., “Microsomal Triglyceride Transfer Protein Enhances Cellular Cholesteryl Esterification by Relieving Product Inhibition”, J. Biol. Chem., Vol. 283, No. 29, pp. 19967-19980 (Jul. 18, 2008), the entire contents of which are incorporated by reference herein.

### EXAMPLES

### Example 1

### Hepatic Expression and Activity of MTP in Conditional MTP Knockout Mice and Its Effect on Tissue Lipid Levels.

In this Example, Mttp^{tm2sgy} Ldlr^{tm1Her} Apob^{tm2Sgy} Tg(Mx1-cre)1Cgn/J mice (n=3) were injected three times with PBS or pIpC. Total RNA was isolated from the liver and used for quantitative RT-PCR of MTP as well as ARPp0 mRNA. The ratio of MTP to ARPp0 in one PBS injected mice was used for normalization. Liver tissues were also used to measure MTP activity. In addition, triglyceride, total, Free and Esterified cholesterol levels were measured in the liver of PBS and pIpC injected animals. (**, p<0.01, ***, p<0.001.)

The results indicate that pIpC injection provides successful deletion of MTP gene as evident by significantly reduced MTP activity in the liver of these mice. MTP gene deletion increased hepatic TG and FC levels, and decreased esterified cholesterol levels.

### Example 2

### Effect of Mttp Gene Deletion on Plasma Lipids and Liver Function Enzymes:

In this Example, conditional MTP deletion was achieved by injecting (n=3) pIpC three times in Mttp^{tm2Sgy} Ldlr^{tm1Her} Apob^{tm2Sgy} Tg(Mx1-cre)1Cgn/J mice on alternate days.

Control mice (n32 3) received PBS injections. Plasma samples were then assayed for total, non-HDL apoB-lipoproteins, and HDL cholesterol and triglyceride levels, respectively. Plasma was also used to measure alanine aminotransferase and aspartate aminotransferase activities. Each measurement was performed in triplicate. The data are representative of 3 independent experiments. The asterisks show statistically significant differences between PBS and pIpC injected mice. (**, p<0.01, ***, p<0.001.)

It can be concluded that MTP gene deletion significantly reduces non-HDL apoB-lipoproteins in the plasma and increases liver enzymes (ALT, AST) in the plasma.

### Example 3

Effect of Efflux Enhancer, T0901317, and Cholesterol Biosynthesis Inhibitor, Lovastatin, on Plasma Lipids and Liver Function Enzymes in PBS and pIpC Injected Animals:

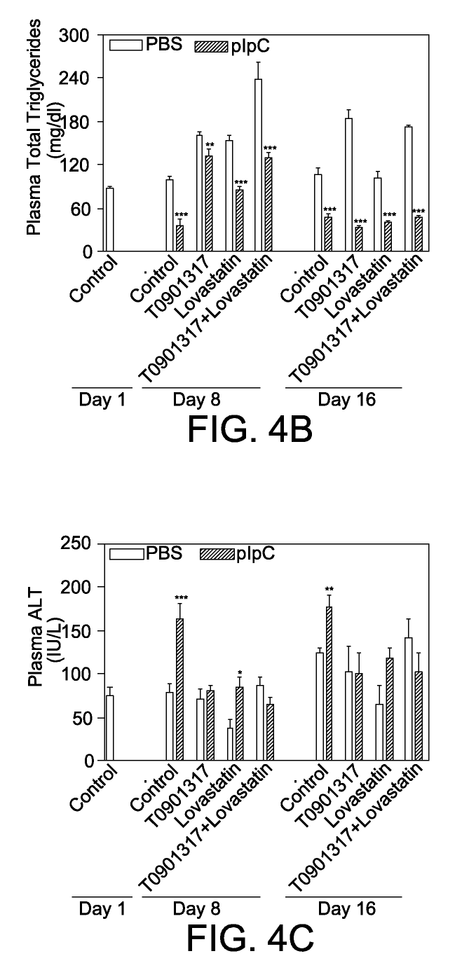

In this Example, blood was collected from tail vein of animals on day 1 of the experiment and used to measure plasma lipid and liver function enzyme levels (Day 1, “Control”). The results of this Example are set forth in FIG. 4.

Conditional MTP deletion was achieved by injecting pIpC three times in Mttp^{tm2Sgy} Ldlr^{tm1Her} Apob^{tm2Sgy} Tg(Mx1-cre)1Cgn/J mice on alternate days. Control group mice (n=2) received either PBS (open bars) or pIpC (closed bars) injections. T0901317, lovastatin or T0901317+lovastatin group animals (n=3) were given 50 mg/Kg of drug by oral gavage every day for 2 weeks. On day 8, blood was again collected to measure changes in plasma lipid and liver function enzyme levels. Mice were sacrificed on day 16 and plasma samples were then assayed for total cholesterol (A), triglyceride (B), ALT (C) and AST (D) levels. Each measurement was performed in triplicate. The asterisks show statistically significant differences between PBS and pIpC injected mice. (*, p<0.05, **, p<0.01, ***, p<0.001.)

The results indicate that MTP gene deleted animals (pIpC injected) have low plasma cholesterol and triglyceride levels. These levels are not significantly altered by T0901317 and lovastatin. MTP gene deletion enhances plasma ALT and AST levels. This increase is not seen in animals fed with T0901317 or lovastain indicating that increases in liver due to MTP gene deletion can be avoided by co-administration of these compounds.

### Example 4

Effect of Efflux Enhancer, T0901317, and Cholesterol Biosynthesis Inhibitor, Lovastatin, on Hepatic mRNA, MTP Activity and Lipid Levels in PBS and pIpC Injected Animals:

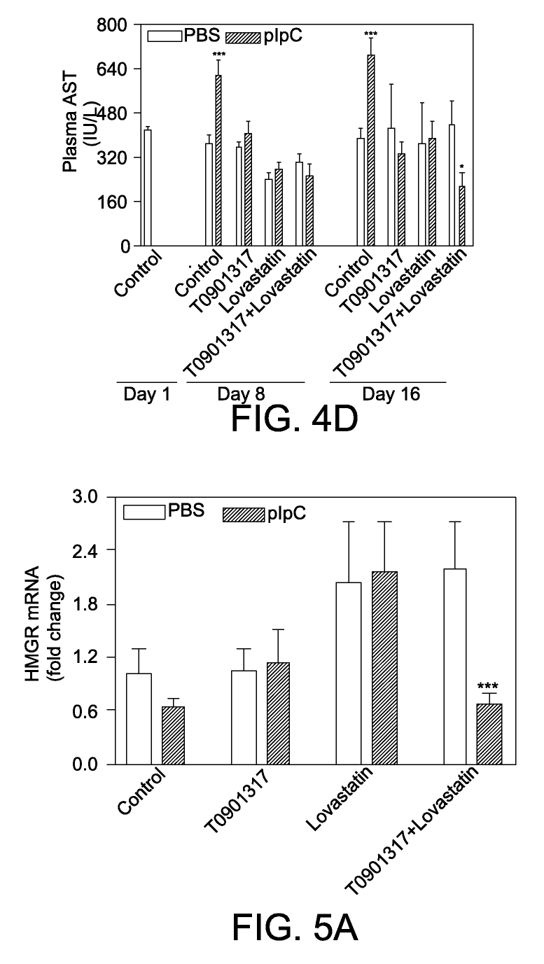

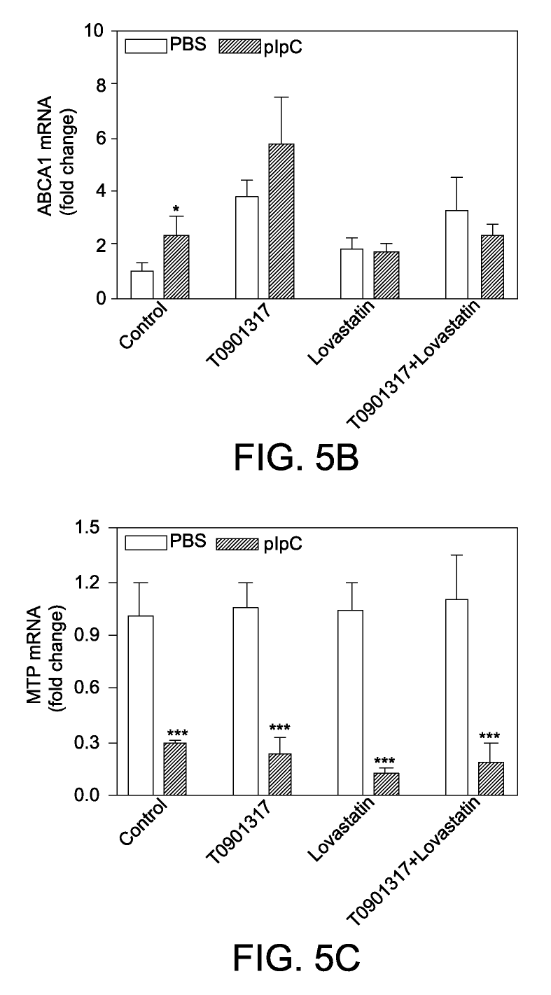

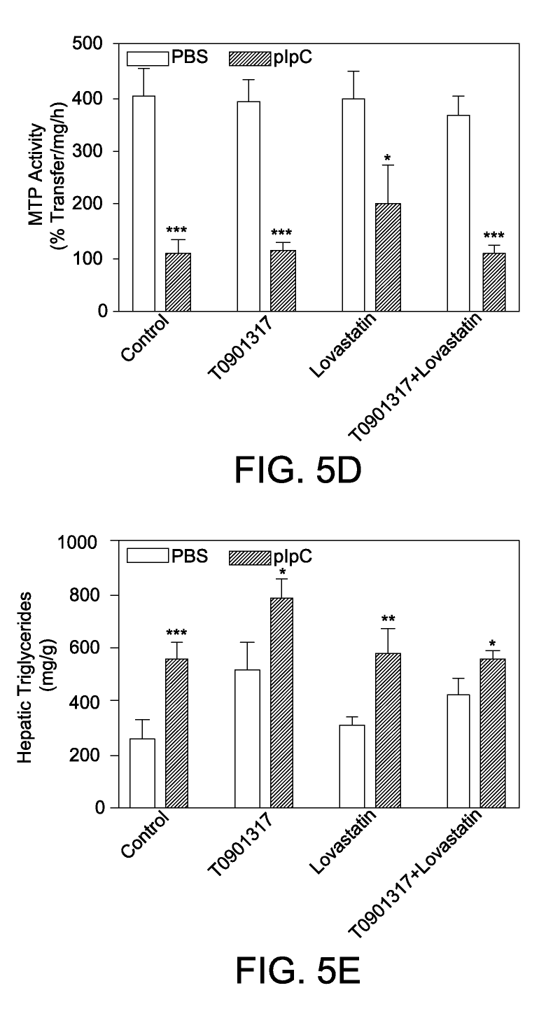

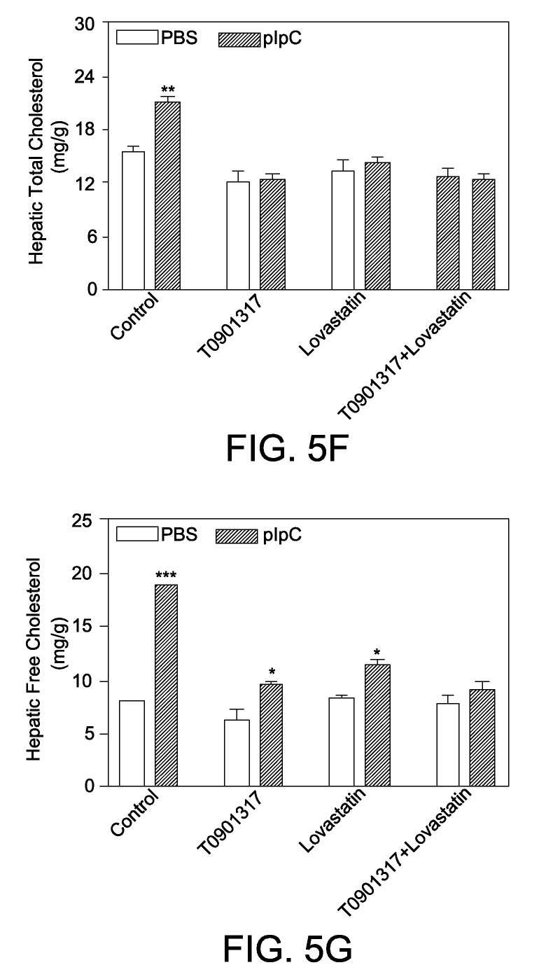

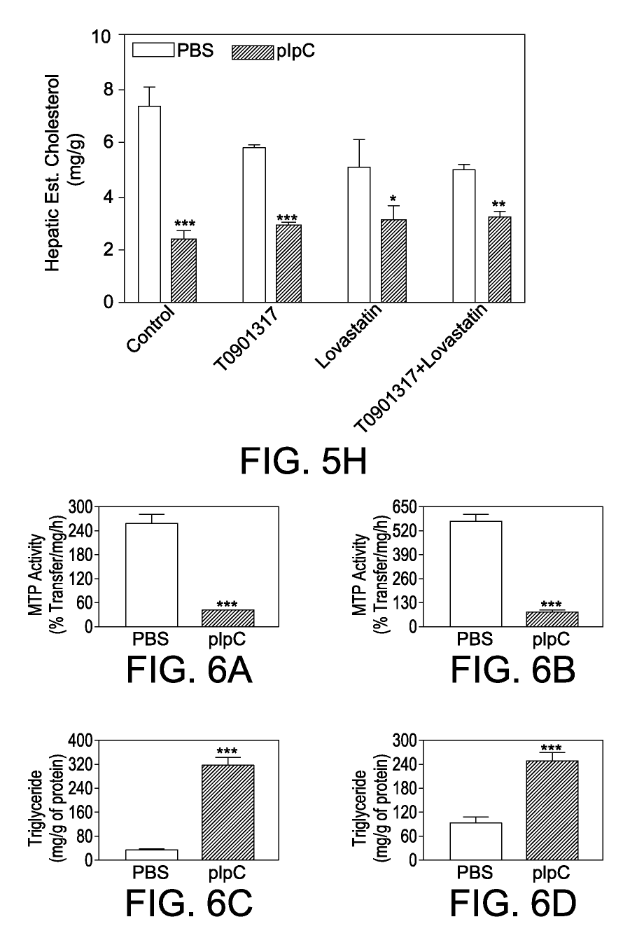

In this Example, total RNA was isolated from the liver of drug administered PBS and pIpC injected mice and used for quantitative RT-PCR of HMG-CoA reductase (FIG. 5, Panel A), ABCA1 (FIG. 5, Panel B), MTP (FIG. 5, Panel C) as well as ARPp0 mRNA. The ratio of candidate gene to ARPp0 in one PBS injected mice was used for normalization. Liver tissues were also used to measure MTP activity (FIG. 5, Panel D). In addition, triglyceride (FIG. 5, Panel E), total (FIG. 5, Panel F), Free (FIG. 5, Panel G) and Esterified (FIG. 5, Panel H) cholesterol levels were measured. (*, p<0.05, **, p<0.01, ***, p<0.001.)

The results indicate that hepatic free cholesterol levels are increased in MTP gene deleted animals. These increases are reduced when animals were also given T0901317 or lovastatin. MTP gene deletion reduces HMG-coA reductase mRNA levels and increases ABCA1 mRNA levels indicating increases in cellular free cholesterol levels. These changes are not seen when animals also received other compounds.

### Example 5

### Role of MTP in the Control of Cholesteryl Ester Biosynthesis.

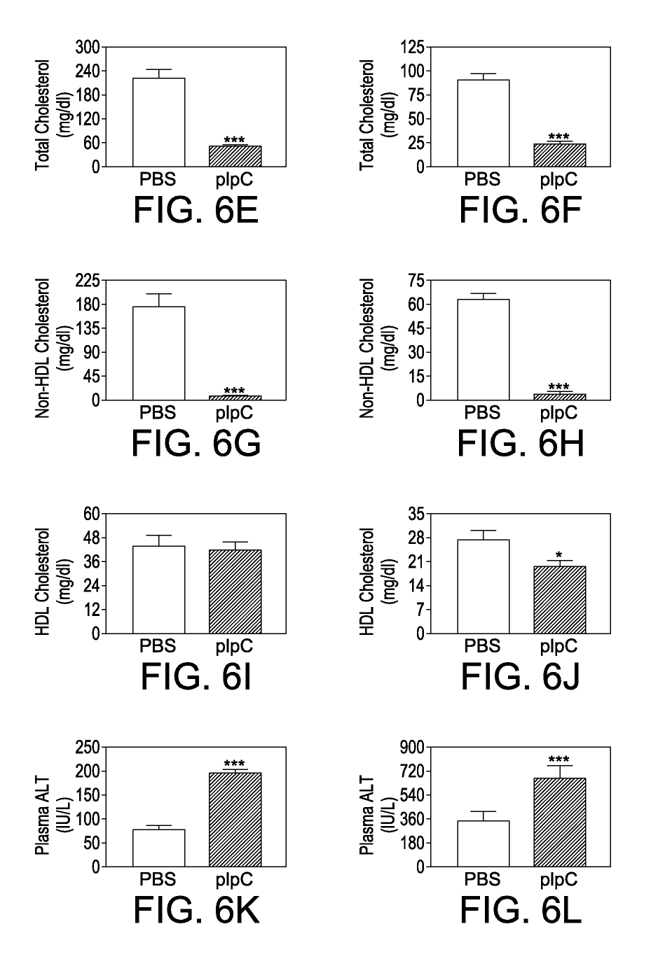

Mttp^{tm2Sgy} Ldlr^{tm1Her} Apob^{tm2Sgy} Tg(Mx1-cre)1Cgn/J mice (stock number 004192) were injected three times on alternate days with either PBS or pIpC. Injection of pIpC is known to activate the Cre recombinase and delete MTP gene in these mice. There was approximately an 80-85% reduction in MTP activity in the intestine and liver of mice injected with pIpC compared to control mice that received PBS. (FIG. 6, A and B). This reduction indicates successful MTP gene deletion.

Triglyceride levels in the intestines and livers were measured and were found to have been increased approximately 8-fold in the intestine (FIG. 6, C) and approximately 3-fold in the liver (FIG. 6, D).Analysis of plasma lipids revealed that the plasma total cholesterol was decreased by 66% (FIG. 6, E), and the triglyceride was decreased by 62% (FIG. 6, F). The decrease in cholesterol and triglyceride was mainly due to reduction in non-HDL apoB-lipoproteins (FIG. 6, G, H). There was no significant change in the HDL cholesterol levels (FIG. 6, I). HDL triglyceride showed small but significant decrease (FIG. 6, J). Plasma alanine aminotransferase and aspartate aminotransferase were significantly increased in the pIpC injected animals (FIG. 6, K, L). Thus, the deletion of the mttp gene increased tissue triglyceride, decreased lipids in apoB-lipoproteins, and elevated liver enzymes in the plasma.

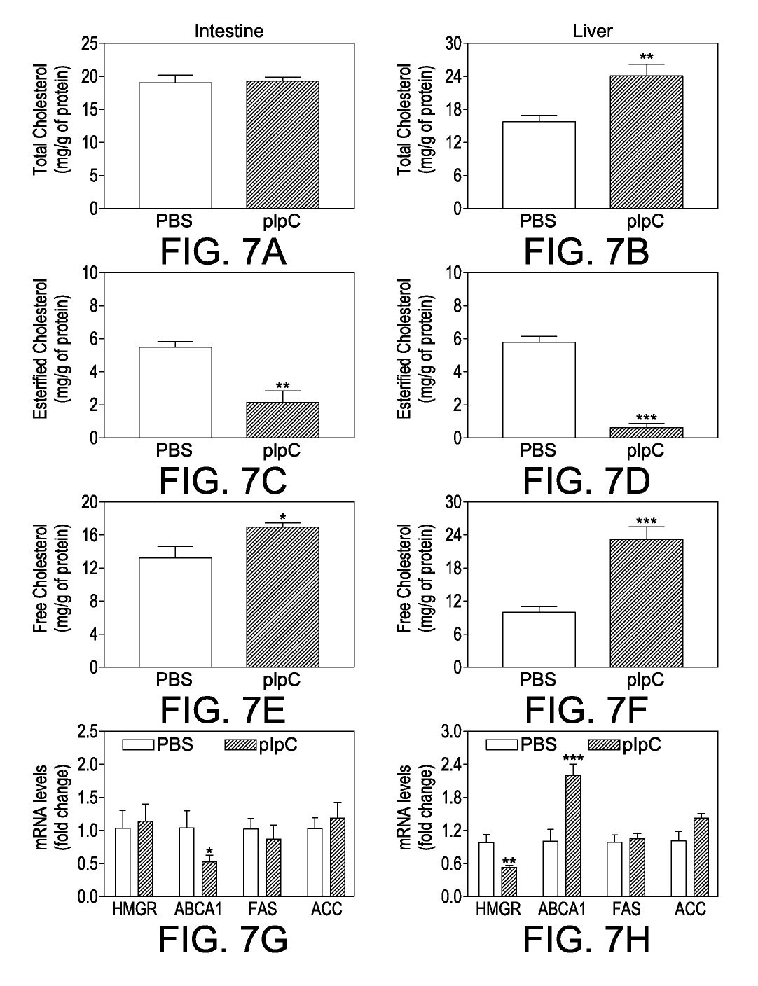

Tissue cholesterol levels were also analyzed. There was found to be no change in the levels of total cholesterol in the intestine (FIG. 7, A), however total cholesterol increased by 52% in the liver of mice injected with pIpC (FIG. 7, B). Similar to the triglycerides as set forth above, it was anticipated that the cholesteryl ester levels would also increase in the intestine and liver of mttp deficient mice. However, unexpectedly, there was a 60% reduction in esterified cholesterol in the intestine (FIG. 7, C) and 90% reduction in the esterified cholesterol in the liver (FIG. 7, D). Additionally, free cholesterol increased by 29% in the intestine (FIG. 7, E) and by 132% in the liver (FIG. 7, F) in mice subjected to conditional mttp gene dilution.

It is known that changes in cellular free cholesterol levels affect genes involved in cholesterol homeostasis. Therefore, changes in mRNA levels of different candidate genes in the intestine (FIG. 7, G) and the liver (FIG. 7, H) were measured. Increases in free cholesterol were associated with decreased HMG-coA reductates and increased ABCA1 mRNA levels in the liver.

These studies indicate that hepatic MTP ablation results in the accumulation of free cholesterol and alterations in sterol response genes. Thus, loss of MTP results in significant increases in tissue triglyceride as well as free cholesterol and substantial decreases in cholesteryl ester levels.

### Example 6

### MTP Gene Deletion and ACAT Activity.

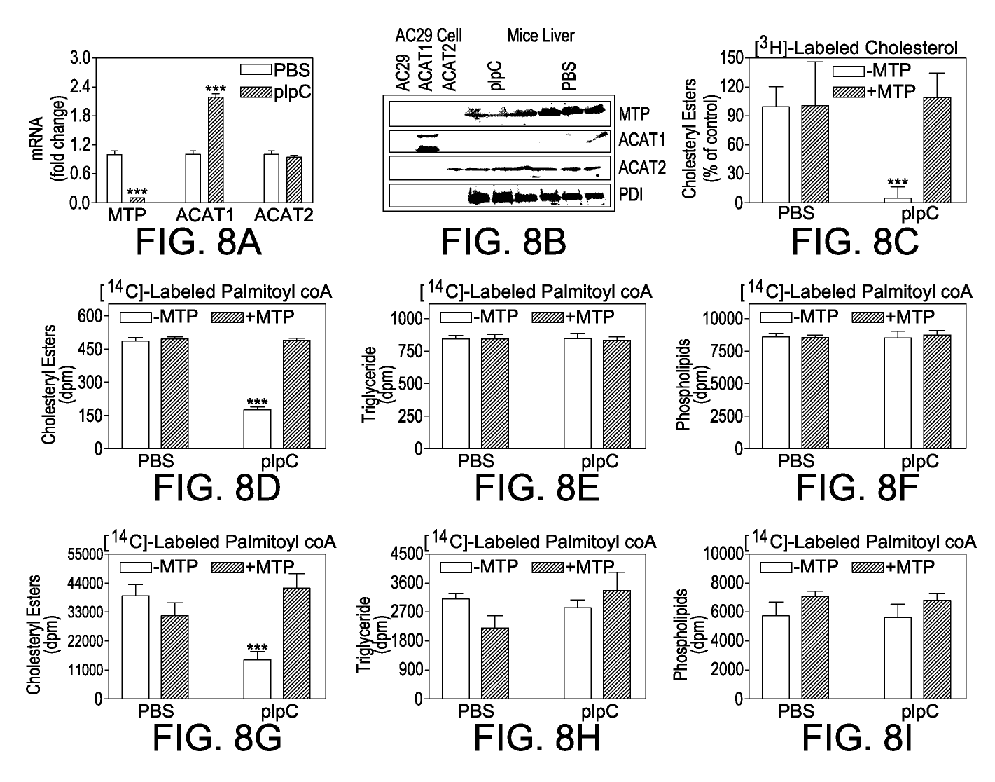

It was hypothesized that MTP may be a chaperone for the enzymes involved in the biosynthesis of cholesteryl esters, as it is for the synthesis of apoB-lipoproteins and CD1d. To examine this, steady state mRNA levels of MTP, ACAT1 and ACAT2 in PBS and pIpC injected mice. As expected, after pIpC injections, MTP mRNA was significantly reduced in the liver (FIG. 8, A) and the intestine (FIG. 8, B). pIpC injections, however, increased hepatic and intestinal ACAT1 mRNA levels but had no effect on ACAT2 mRNA levels (FIG. 108 A, B). At this time, we have no explanation for the increases in ACAT1 mRNA levels in pIpC-injected mice. pIpC injection does not increase ACAT1 mRNA in C57B116J mice. These data show that deletion of mttp enhances steady state ACAT1 mRNA levels in the liver and intestine without affecting ACAT2 levels.

The synthesis of cholesteryl esters were used to elucidate reasons for decreased cellular cholesteryl ester levels in MTP deficient tissues. Cholesteryl ester synthesis was measured using [³H] cholesterol and hepatic microsomes isolated from mice injected with PBS or pIpC in the presence or absence of purified MTP (FIG. 8, C). Addition of purified MTP did not affect cholesteryl ester synthesis by microsomes isolated from control PBS injected mice (FIG. 8, C, first pair of bars). Microsomes isolated from pIpC-injected animals showed a 96% decrease in ACAT activity compared to microsomes isolated from PBS injected animals (FIG. 8, C, open bars). There was no reduction in mRNA levels of ACAT1 and ACAT2. Western blot analysis also showed no significant decreases in the protein levels of these enzymes.

It was therefore concluded that MTP deficiency does not affect ACAT expression. It was hypothesized that if MTP is essential for optimal esterification, then supplementation of purified MTP to these microsomes may enhance cholesterol esterification. Indeed, addition of purified MTP to these microsomes completely restored cholesteryl ester synthesis (FIG. 8, C, solid bars) indicating that reduced cholesterol ester synthesis was related to MTP deficiency and was not due to a reduction in ACAT enzymes. Similar results were obtained when radio-labeled palmitoyl-coA was used to study microsomal cholesteryl esterification (FIG. 8, D). Supplementation of control microsomes with purified MTP had no effect on cholesteryl ester synthesis (FIG. 8, D, first pair of bars). MTP gene deletion decreased the esterification of cholesterol (FIG. 8, D: open bars) and addition of purified MTP to these microsomes restored cholesteryl ester synthesis (FIG. 8, D, solid bars). MTP gene deletion as well as supplementation of purified MTP had no effect on the synthesis of triglycerides (FIG. 8, E) and phospholipids (FIG. 8, F) by hepatic microsomes.

Consideration was then given to the possibility that cholesterol in the membrane might be limiting. To circumvent this, cholesterol ester synthesis was studied by providing cholesterol as part of cyclodextran complexes. Again, results were similar to those seen in reactions not supplemented with additional cholesterol (FIG. 8, D). Synthesis of cholesterol esters was significantly higher under these conditions (FIG. 8, G, first pair of bars) compared with conditions when no additional cholesterol was provided (FIG. 8, D), consistent with the idea that higher cholesterol levels are allosteric activators of cholesteryl ester synthesis. Cholesteryl ester synthesis by the microsomes isolated from pIpC-injected mice was significantly lower (FIG. 8, G, open bars). This deficiency could be corrected by the addition of purified MTP (FIG. 8, G, solid bars).

Under the same conditions, the synthesis of triglycerides (FIG. 8, H) and phospholipids (FIG. 8, I) was unaffected by the deficiency or supplementation of MTP. Thus, MTP gene deletion does not reduce the synthesis of triglycerides and phospholipids, but specifically decreases cholesteryl ester synthesis. The decline in cholesteryl ester synthesis can be restored by the addition of purified MTP. It was therefore concluded that MTP is not required for the biosyntheses of enzymes involved in cholesteryl ester synthesis, but it modulates the cholesteryl esterification reaction by a hitherto unrecognized mechanism.

### Example 7

### Effect of MTP and ACAT Antagonists on Cellular Cholesteryl Ester Levels.

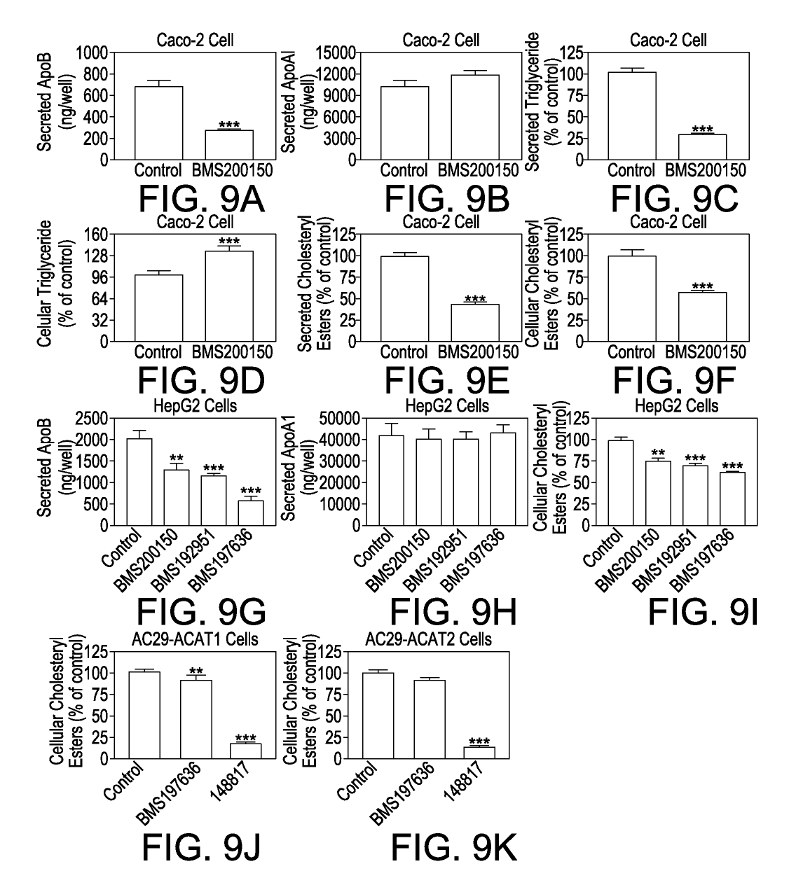

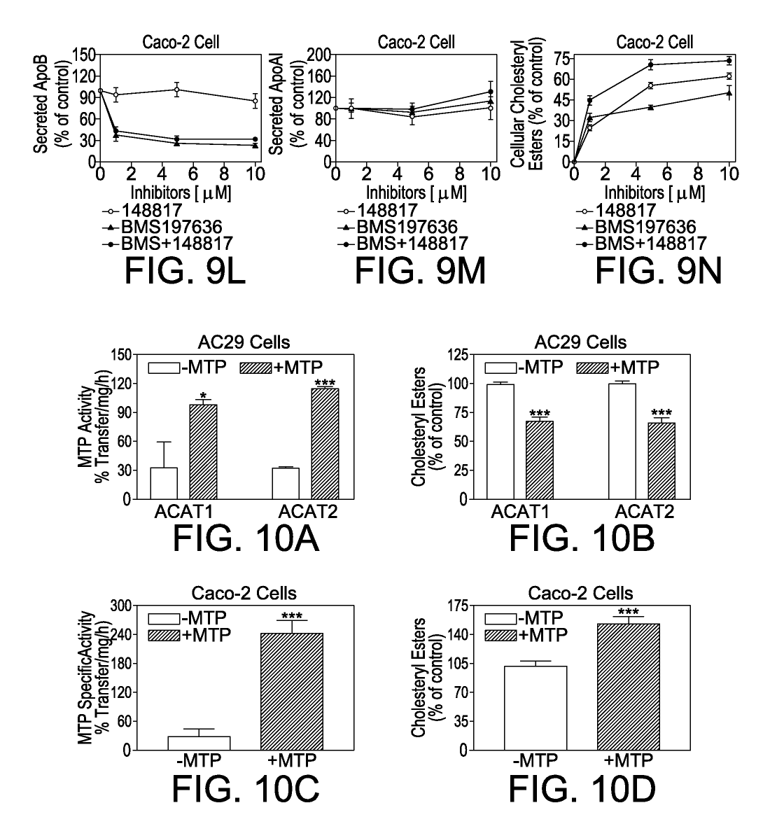

It is known that deletion of mttp gene results in significant alterations in cellular lipid homeostasis. To eliminate mechanisms involving homeostatic changes due to long term mttp gene deletion, the effect of acute inhibition of MTP activity using specific chemical antagonists on cellular cholesteryl ester synthesis was measured. As expected, MTP inhibitors decreased apoB secretion (FIG. 9, A), had no effect on apoAI secretion (FIG. 9, B), decreased triglyceride secretion (FIG. 9, C), increased cellular triglycerides (FIG. 9, D), and decreased cholesteryl ester secretion (FIG. 9, E) in differentiated Caco-2 cells. Instead of an expected increase in cellular radio-labeled cholesteryl esters due to their decreased secretion, there was a 60% reduction in their cellular levels (FIG. 9, F).

To rule out the possibility that the effect on cellular cholesterol esterification was unique to the inhibitor and Caco-2 cells used, HepG2 cells were labeled with [³H] cholesterol and then treated with different MTP inhibitors (1 pM). As expected, all the MTP inhibitors reduced apoB secretion (FIG. 9, G) and had no effect on apoA1 secretion (FIG. 9, H).

Next, the effect of these inhibitors on cellular cholesteryl ester synthesis was examined. Newly synthesized cellular cholesteryl ester levels were significantly reduced in cells treated with different antagonists (FIG. 9, I). These studies showed that chemical inhibition of MTP also leads to decreased cholesteryl ester synthesis in HepG2 cells. To confirm further that the effect of MTP antagonists was specific to MTP and that they did not inhibit ACAT enzymes, the effect of MTP antagonists in AC29 cells that do not express MTP but stably express ACAT1 or ACAT2 was examined. In these cells, MTP inhibitor BMS 197636 had no effect on cellular cholesteryl ester synthesis (FIG. 9, J and K). However, incubation of these cells with ACAT inhibitor 148817 reduced radio-labeled cholesteryl esters by 83-87% (FIG. 9, J, K). These studies demonstrate that MTP inhibitors do not affect ACAT activities.

The genetic ablation and chemical inhibition studies described above clearly showed that MTP affects cholesteryl esterification without affecting the ACAT enzyme activities. Thus, it was hypothesized that MTP (BMS 197636) and ACAT (1 488 17) antagonists would inhibit cellular esterification by independent mechanisms and their effect would be additive. To test this hypothesis, Caco-2 cells were treated with different amounts of these inhibitors, alone or in combination, and the inhibition was compared with cells that were not exposed to any inhibitors (FIG. 9, L-N). Increasing concentrations of BMS 197636 decreased apoB secretion (FIG. 9, L). In contrast, ACAT inhibitor had no effect on apoB secretion. The combined effect of both the inhibitors was similar to that of MTP inhibitor alone indicating that ACAT inhibitor, alone or in combination, had no effect on apoB secretion. ApoAI secretion was resistant to both the inhibitors (FIG. 9, M). Individually, both the inhibitors at 1 pM concentration inhibited cellular esterification of [³H] cholesterol to similar extents (25 +/−2% and 32 +/−3% inhibition) (FIG. 9, N). These inhibitors in combination reduced cholesteryl ester synthesis by 45 +/−4% (FIG. 9, N). Thus, MTP and ACAT act in tandem and facilitate cellular cholesteryl esterification.

### Example 8

MTP and apoB in the Synthesis of Cholesteryl Esters.

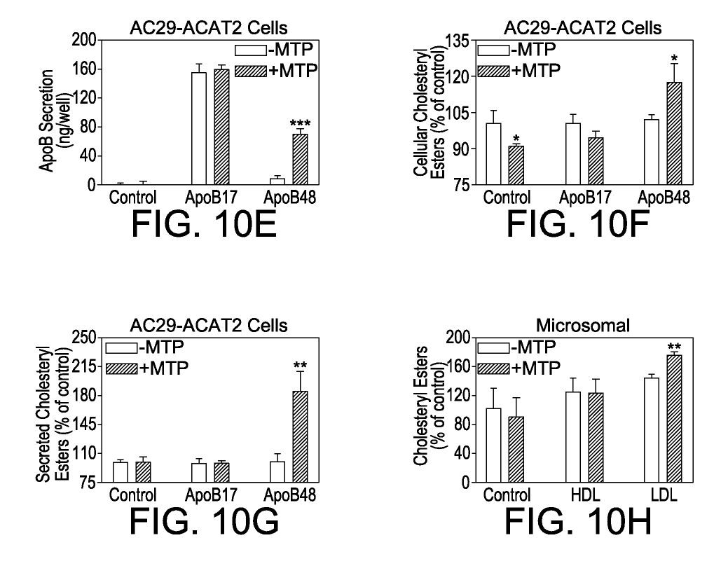

It was hypothesized that MTP overexpression would lead to increased cellular cholesteryl esterification. To test this hypothesis, human MTP was expressed in AC29-ACAT1 and AC29-ACAT2 cells. These cells have been generated by stable transfection of AC29 cells, lack cholesteryl ester synthesizing enzymes, with ACAT1 and ACAT2 enzymes. As expected, MTP expression increased triglyceride transfer activity by 3- to 3.5-fold in both the cell lines (FIG. 10, A). The esterification of [³H] cholesterol in these cell lines was then measured. Instead of an expected increase in the levels of cholesteryl esters, lower cholesteryl ester levels were found in these cells (FIG. 10, B). To explain these unanticipated results, similar experiments were repeated in non-differentiated Caco-2 cells. Expression of MTP increased triglyceride transfer activity by 8 to 9-fold (FIG. 10, C) and cellular cholesteryl ester levels (FIG. 10, D) by 50% in these cells. These studies show that MTP expression increases cellular cholesteryl esterification in Caco-2 cells, but not in AC29 cells expressing ACAT enzymes.

The data suggests that AC29 cells lack another factor required for maximal cellular esterification and that this factor is present in Caco-2 cells. One major difference between these two cell lines is that Caco-2 cells synthesize and secrete apoB-lipoproteins, whereas AC29 cells do not. Thus, the question was whether apoB is also required for enhanced cellular cholesteryl esterification. AC29-ACAT2 cells were transfected with plasmids expressing either human apoB 17 or apoB48 with or without human MTP expressing plasmids. Secretion of apoB 17 does not require lipidation by MTP and can be secreted in the absence of MTP (FIG. 10, E) consistent with several published studies. However, apoB48 secretion requires lipidation by MTP and is not secreted in the absence of MTP. As expected, there was a significant increase in the secretion of apoB48 when the cells were co-transfected with MTP (FIG. 10, E). Expression of apoB 17 or apoB48 without MTP had no significant effect on the cellular (FIG. 10, F, open bars) and secreted (FIG. 10, G, open bars) cholesteryl ester levels. When the cells were transfected with MTP alone there was a reduction in cellular cholesteryl ester levels (FIG. 10, F, control) as observed before (FIG. 10, B). Cells expressing MTP and apoB 17 synthesized and secreted similar amounts of cholesteryl esters as control cells (FIG. 10, F-G, apoB 17). However, when AC29-ACAT2 cells were co-expressing both MTP and apoB48, there was approximately 17% increase in cellular cholesteryl esters (FIG. 10, F, apoB48) and 80% increase in secreted cholesteryl ester levels (FIG. 10, G, apoB48). These studies indicate that both MTP and apoB are required for optimal cellular cholesteryl ester synthesis.

This was confirmed by studying the effect of apoB and MTP on in vitro cholesteryl esterification using microsomes isolated from ACAT2 cells (FIG. 10, H). When microsomes were incubated with purified MTP alone, there was no change in cholesteryl ester synthesis (FIG. 10, H, control). Similarly, incubation of microsomes with human HDL in the presence or absence of purified MTP also did not increase the cholesteryl ester synthesis. However, when these microsomes were incubated with LDL in the presence of purified MTP, there was a significant increase in cholesteryl esterification (FIG. 10, H). These studies show that the transfer of cholesteryl esters by MTP to apoB lipoproteins enhances cholesteryl ester biosynthesis.

### Example 9

### MTP Modulation of Cholesteryl Esterification.

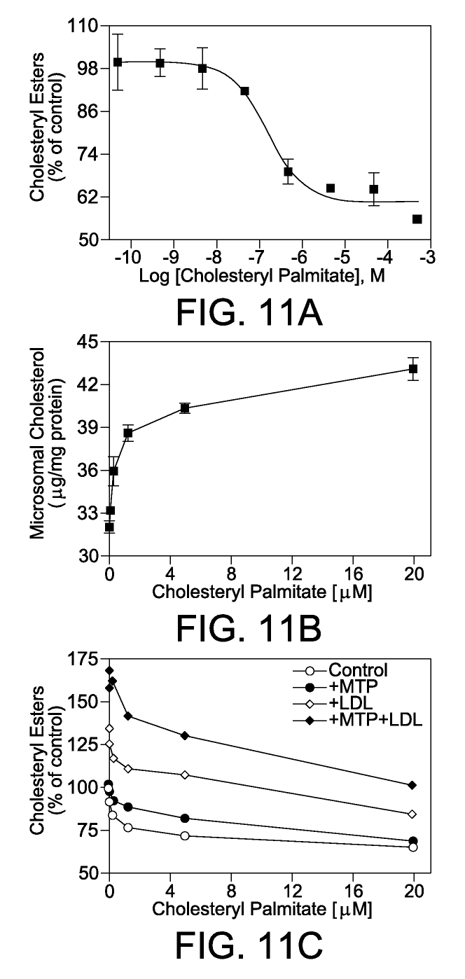

The effect of increasing amounts of cholesteryl palmitate on the esterification of [³H]-cholesterol by liver microsomes was measured. Increasing amounts of cholesteryl palmitate reduced cholesteryl ester synthesis (FIG. 11, A). At 0.5 μM cholesteryl palmitate, cholesteryl ester synthesis was inhibited by 32% (FIG. 11, A). From these experiments, it was not clear whether cholesteryl palmitate was inhibiting cholesteryl ester synthesis by partitioning into the membrane. To determine if microsomal membrane enrichment with cholesteryl palmitate was necessary for decreased cholesteryl ester synthesis, liver microsomes were first incubated with different concentrations of cholesteryl palmitate for 30 min, centrifuged, and cholesterol enrichment of microsomes was determined. Incubation of microsomes with increasing amounts of cholesteryl palmitate resulted in concentration dependent enrichment with cholesterol until approximately 1.25 μM cholesteryl palmitate (FIG. 11, B). Thereafter, there was no significant increase in the enrichment indicating saturation.

Next, the effect of cholesteryl palmitate enrichment of microsomes on cholesteryl ester synthesis in the presence and absence of MTP and LDL was examined (FIG. 11, C). Esterification of cholesterol decreased with increased enrichment of microsomes with cholesteryl palmitate (FIG. 11, C, open circles). As seen before, MTP had no effect on cellular cholesteryl ester synthesis in control, un-enriched microsomes (FIG. 11, C, 0 pM, compare control with +MTP). The amounts of cholesteryl esters synthesized in the presence of MTP decreased with enrichment of microsomes with cholesteryl palmitate (FIG. 11, C, solid circles) similar to that seen in control microsomes (open circles). In these experiments, small endogenous apoB-lipoproteins present as contaminants in microsomal preparations are perhaps sufficient to provide basal cholesterol ester synthesis. However, when control microsomes were supplemented with LDL, there was a significant 35% increase in cholesteryl ester synthesis (FIG. 11, C, 0 pM, open diamonds). Enrichment of these microsomes with cholesteryl palmitate reduced the extent of cholesteryl ester synthesis in the presence of LDL. Furthermore, when control microsomes were incubated with both MTP and LDL there was a significant enhancement (69% of control) in the synthesis of cholesteryl esters (FIG. 11, C, 0 μM, solid diamonds). Again, the extent of cholesteryl ester synthesis under these conditions was reduced when microsomes were enriched with cholesteryl palmitate (FIG. 11, C, solid diamonds).

These studies indicate that enrichment of microsomes with cholesteryl palmitate inhibits cholesteryl ester synthesis. This inhibition is relieved in the presence of MTP and LDL. This data suggests that MTP by transferring cholesteryl esters from microsomal membranes to apoB-lipoproteins relieves product inhibition and enhances cholesteryl ester synthesis.

What is claimed is:

1. A composition comprising:
a. An MTP inhibitor; and
b. At least one of a cholesterol efflux enhancer and a cholesterol synthesis inhibitor.

2. The composition of claim 1, further comprising a bile acid synthesis enhancer.

3. The composition of claim 1, comprising a cholesterol efflux enhancer and a cholesterol synthesis inhibitor.

4. The composition of claim 3, further comprising a bile acid synthesis enhancer.

5. A composition for treating high plasma lipid levels, comprising:
a. A therapeutically effective amount of at least one MTP inhibitor;
b. A therapeutically effective amount of at least one of:
i. at least one cholesterol efflux enhancer and
ii. at least one cholesterol synthesis inhibitor; and
c. A biologically compatible carrier.

6. The composition of claim 5, further comprising a therapeutically effective amount of at least one bile acid synthesis enhancer.

7. The composition of claim 5, comprising a therapeutically effective amount of a cholesterol efflux enhancer and a therapeutically effective amount of a cholesterol synthesis inhibitor.

8. The composition of claim 7, further comprising a therapeutically effective amount of a bile acid synthesis enhancer.

9. A method of treating individuals comprising the steps of:
a. Providing a composition comprising:
i. an MTP inhibitor; and
ii. at least one of a cholesterol efflux enhancer and a cholesterol synthesis inhibitor; and
b. Administering a therapeutically effective amount of said composition to said individual.

10. The method of claim 9, wherein said composition further comprises a bile acid synthesis enhancer.

11. The method of claim 9, wherein said composition comprises a cholesterol efflux enhancer and a cholesterol synthesis inhibitor.

12. The method of claim 11, wherein said composition comprises a bile acid synthesis enhancer.

13. A method of screening potential MTP inhibitors for administration to an individual comprising the steps of:
a. measuring a first free-cholesterol level of at least one liver or intestine derived cell of an individual;
b. administering a candidate MTP inhibitor to cell;
c. measuring a second free-cholesterol level of said cell; and
d. determining whether said second free-cholesterol level is greater than said first free-cholesterol level;
wherein increase of free-cholesterol level is indicative of an effective MTP inhibitor for said individual.

14. A method of screening potential cholesterol efflux enhancers for administration to an individual comprising the steps of:
a. taking a cell sample from said individual;
b. inhibiting MTP from said cell sample to accumulate a first free-cholesterol count;
c. measuring said first free-cholesterol count in said cell sample;
d. administering a candidate free-cholesterol efflux enhancer to said cell sample;
e. measuring a second free-cholesterol count in said cell sample; and
f. determining whether said second free-cholesterol count is lower than said first free-cholesterol count;
wherein decrease of free-cholesterol count is indicative of an effective free-cholesterol efflux enhancer for said individual.
